(19) United States
(12) Patent Application Publication
(10) Pub. No.: US 2014/0045724 A1
(43) Pub. Date: Feb. 13, 2014

(54) FORMULATION SCREENING METHODS, APPARATUSES FOR PERFORMING SUCH METHODS AND FORMULATIONS FORMED BY SUCH METHODS

(71) Applicant: Jon Selbo, West Lafayette, IN (US)

(72) Inventor: Jon Selbo, West Lafayette, IN (US)

(21) Appl. No.: 13/964,986

(22) Filed: Aug. 12, 2013

Related U.S. Application Data
(60) Provisional application No. 61/681,797, filed on Aug. 10, 2012.

(51) Int. Cl. G01N 33/15

(52) U.S. Cl. CPC G01N 33/15
USPC 506/12

(57) ABSTRACT

A method of screening for candidate compound-excipient combinations comprises dosing a compound into each receptacle of a collection of receptacles, dosing a first set of excipients and a second set of excipients into the receptacles wherein the dosing creates varying combinations of solutions of a first and second excipients within the receptacles, analyzing each receptacle for the presence of a precipitate, and classifying the receptacles based on the presence of a precipitate.

### RELATED APPLICATIONS

This application claims priority to U.S. Provisional Patent Application No. 61/681,797 having a filing date of Aug. 10, 2012, which is incorporated herein by reference in its entirety.

### BACKGROUND OF THE INVENTION

Generally speaking, the present application relates to an active pharmaceutical ingredient (API) liquid formulation screening tool to increase solution concentration or useful supersaturation and enable pre-clinical and early clinical evaluation of low solubility APIs. More specifically, the present application relates to a semi-automated screening approach that utilizes a high throughput robotic liquid dispensing platform in conjunction with a microplate reader and polarized light microscope (PLM) to recommend appropriate excipients that may have synergistic effects in increasing both the solubility of an active pharmaceutical ingredient and stabilizing the supersaturation for a useful period of time.

By 1995, it was reported that nearly 70% of all new molecular entities entering pharmaceutical development were Biopharmaceutics Classification System (BCS) class II, meaning they have low solubility and high permeability. These candidates often have delivery limitations due to poor solubility and are generally defined as having dissolution rate limited absorption. For these compounds, one way to increase exposure is by increasing the concentration of drug in the solution at the absorption site. This can be done in a number of ways such as by salt modification, form modification (e.g. amorphous forms), or by the use of surfactants that increase the solubility. More common in early preclinical testing is the use of supersaturated solutions that maintain a metastable API concentration through the prevention of in situ nucleation or crystal growth by the addition of polymers.

For compounds that exhibit dissolution rate limited absorption, the most important parameter for improving bioavailability is increasing the concentration of the API in the absorption window. In early in vivo testing, this often means solution/suspension dosing with high levels of excipients that may not be suitable for later preclinical development. The present application offers a screen that quickly tests the interaction of multiple excipients with the API in a desired vehicle (e.g. water, buffer, etc.) to determine possible synergistic effects for increasing both solubility and supersaturation.

It has been reported that approximately 40% of all discovered drugs have delivery limitations due to either poor solubility or poor bioavailability (Ping Li and Luwei Zhao, Developing early formulations: Practice and perspective, International Journal of Pharmaceutics 341 (2007) 1-19.) One of the means to increase solubility of compounds—and therefore increase bioavailability for compounds that exhibit dissolution limited absorption by supersaturating the solvent system through an appropriate combination of excipients in order to stabilize the active pharmaceutical ingredient (“API”) and prevent its in situ nucleation/recrystallization. The resulting combinations, including those where no or reduced precipitation is observed, may be suitable candidates for continued development.

The present application offers a semi-automated method for developing aqueous formulations with improved API solution concentration and useful supersaturation. The present application demonstrates the capability of a semi-automated preformulation screen for quickly identifying multiple excipient-surfactant and excipient-inclusion compound combinations that will increase the solubility of an API in an aqueous medium and determine synergistic supersaturation effects.

### BRIEF SUMMARY OF THE INVENTION

The present application relates to an active pharmaceutical ingredient (API) liquid formulation screening method to increase solution concentration or useful supersaturation and enable pre-clinical and early clinical evaluation of low solubility APIs. More specifically, the present application relates to a semi-automated screening method that utilizes a high throughput robotic liquid dispensing platform in conjunction with a microplate reader and polarized light microscope (PLM) to recommend appropriate excipients that may have synergistic effects in increasing both the solubility of an active pharmaceutical ingredient and stabilizing the supersaturation for a useful period of time. The present application also relates to apparatuses for carrying out such a method and formulations formed by such a method.

The present method of screening for candidate compound-excipient combinations comprises dosing a compound into each receptacle of a collection of receptacles; dosing a first set of excipients and a second set of excipients into said receptacles wherein said dosing creates multiple combinations of solutions of the first excipient set and the second excipient set within said receptacles; analyzing each receptacle for the presence of a precipitate; and classifying the receptacles based on the presence of a precipitate.

In certain embodiments, the compound can be an active pharmaceutical ingredient, a nutraceutical, or an agricultural active substance.

In certain embodiments, the receptacle can be a well plate, a vial, or a test tube.

In an embodiment the compound can be dosed as a solid. In another embodiment, the compound can be dosed in an organic solvent. In yet another embodiment, the compound and organic solvent can form a solution.

In an embodiment, the receptacles can be classified as either having precipitate or not having precipitate. In another embodiment, the receptacles can be classified based on the relative amount of precipitate present.

In an embodiment the method can further comprise the step of removing the organic solvent prior to dosing with the first and second excipient sets.

In certain embodiments the at least one of the set of excipients can include a set of polymers, a set of surfactants or a set of inclusion compounds.

In an embodiment, the concentration of said first and second excipient sets are in ratios relative to their amounts dosed in the ratios of high/high, high/low, low/high and low/low combinations of said first excipient/second excipient sets wherein the high concentration and the low concentrations are fixed for a set of receptacles.

In certain embodiments, the precipitation is analyzed in said receptacles using light obscuration, visual observation or optical microscopy.

In an embodiment, the solvent is removed by evaporation.

In an embodiment, the first and second excipient sets are dosed in an aqueous solution, such as an aqueous ethanol solution.

In an embodiment, the compound is supersaturated after dosing with the excipients.

In an embodiment, supersaturation is obtained by increasing and then decreasing the temperature of said receptacle.

In certain embodiments, the receptacle is shaken, centrifuged or sonicated after dosing.

In one embodiment the method further comprises performing a concentration gradient analysis comprising; dosing said compound into a second set of receptacles wherein said compound is dosed from a minimum to a maximum amount of compound; dosing into each set of receptacles a first and second excipient set selected from the ratios identified in claim 14; analyzing each receptacle for the presence of a precipitate; classifying the receptacles based on the presence of a precipitate to identify a solubility limit of the composition in the receptacle.

In certain embodiments, the receptacle can be a well plate, a vial, or a test tube.

In an embodiment the compound can be dosed as a solid. In another embodiment, the compound can be dosed in an organic solvent. In yet another embodiment, the compound and organic solvent can form a solution.

In an embodiment, the method further comprises the step of identifying a combination with the highest solubility limit or identifying a combination based upon the solubility limit.

In an embodiment the method can further comprise the step of removing the organic solvent prior to dosing with the first and second excipient sets.

In certain embodiments, the precipitation is analyzed in said receptacles using light obscuration, visual observation or optical microscopy.

In an embodiment, the first and second excipient sets are dosed in an aqueous solution, such as an aqueous ethanol solution.

In an embodiment, the compound is supersaturated after dosing with the excipients.

In an embodiment, supersaturation is obtained by increasing and then decreasing the temperature of said receptacle.

In certain embodiments, the receptacle is shaken, centrifuged or sonicated after dosing.

An apparatus for screening active pharmaceutical ingredient liquid formulations comprises a liquid handling robot for dosing a fixed concentration of the active pharmaceutical ingredient into a first wellplate wherein the fixed concentration of active pharmaceutical ingredient is dosed into each well and dosing a set of first excipients and a set of second excipients into said wells wherein said dosing creates varying combinations of a first and second excipient within said wells; and a light obscuration device or an optical microscope for checking precipitation in each of said compartments after a period of time to identify candidate excipient combinations based on a lack of precipitation after said period of time.

The apparatus can further comprise said liquid handling robot being effective for dosing said active pharmaceutical ingredient into a second wellplate wherein said active pharmaceutical ingredient is dosed in multiple sets from a minimum to maximum concentration by incremental increases and dosing each of said candidate excipient combinations into said sets at fixed concentrations wherein one candidate excipient combination is dosed into a set and one control set is left with only active pharmaceutical ingredient; and said light obscuration device or optical microscope being effective for checking precipitation in each of said compartments after a period of time to identify upper solubility limits for each candidate excipient combination.

These and other advantages and novel features of the present invention, as well as details of illustrated embodiments thereof will be more fully understood from the following description of the drawings.

### BRIEF DESCRIPTION OF SEVERAL VIEWS OF THE DRAWINGS

[Not Applicable]

### DETAILED DESCRIPTION OF THE INVENTION

The present application relates to methods for screening candidate compound excipient combinations. The present application relates to an active pharmaceutical ingredient (API) liquid formulation screening method to increase solution concentration or useful supersaturation and enable pre-clinical and early clinical evaluation of low solubility APIs. More specifically, the present application relates to a semi-automated screening method that utilizes a high throughput robotic liquid dispensing platform in conjunction with a microplate reader and polarized light microscope (PLM) to recommend appropriate excipients that may have synergistic effects in increasing both the solubility of an active pharmaceutical ingredient and stabilizing the supersaturation for a useful period of time. The present application also relates to apparatuses for carrying out such a method and formulations formed by such a method.

In one aspect of the present method for screening candidate compound excipient combinations comprises dosing a compound into each receptacle of a collection of receptacles; dosing a first set of excipients and a second set of excipients into said receptacles wherein said dosing creates varying combinations of solutions of a first and second excipients within said receptacles; analyzing each receptacle for the presence of a precipitate; and classifying the receptacles based on the presence of a precipitate.

The compound can be selected from any number of classes of compounds including, but not limited to, an active pharmaceutical ingredient, a nutraceutical, or an agricultural active substance. The compound can be dosed as a solid. The compound can also be dosed in an organic solvent. The compound and organic solvent together can be in the form of a solution. In another embodiment, the compound may be dosed in aqueous solution.

The receptacle can be a well plate, a vial, or a test tube. The receptacles can be classified as either having precipitate or not having precipitate. They can also be classified based on the relative amount of precipitate present. Receptacles include, but are not limited to, well plates, vials, and test tubes. In a further embodiment the compound is dosed into a receptacle by administering the compound in a solid form into the receptacle. In another embodiment the compound is dosed into a receptacle by administering the compound as a solution in an organic solvent and the organic solvent is subsequently removed. The method can further comprise the step of removing the organic solvent prior to dosing with the first and second set of excipients. In further embodiments the organic solvent may be removed by evaporation. Suspensions may also be dosed in other embodiments

In some embodiments, the combination comprises a first set excipients which includes, but is not limited to, a polymer or a set of polymers and a second set of excipients which includes, but is not limited to, a surfactant or a set of surfactants. In another embodiment the combination comprises at least one set of excipients which includes, but is not limited to, a set of inclusion compounds. Excipient sets may comprise a single excipient or multiple excipients. Examples of possible excipients include KLUCEL® Hydroxypropylcellulose (HPC), Hydroxypropylmethylcellulose (HPMC), POLYOX™ (WSR N10 LEO NF), Kollidon® polyvinylpyrrolidone-vinyl acetate 64 (PVPVA64), Plasdone® polyvinylpyrrolidone K-29/32 (PVPK29/32), Plasdone® polyvinylpyrrolidone K-90 (PVPK90), polyethylene glycol 4000 (PEG4000), Cetrimide, sodium dodecyl sulfate (SDS), potassium laurate (PL), TWEEN® 20, TWEEN® 60, TWEEN® 80, Cremophore® RH40, sucrose monolaurate (SML), α-tocopheryl polyethylene glycol 1000 succinate (TPGS), PEG-PPG-PEG block copolymer (Mn=1100) (PEG-PPG-PEG), Chemical Macat® LB lauryl dimethyl betaine (LDMB) and 2-hydroxypropyl-β-cyclodextrin (HPβCD).

In many embodiments of the invention, two sets of excipients are selected for each screen and two different concentrations are used for each excipient set resulting in four different possible concentrations of excipients per receptacle referred to herein as “high/high”; “high/low”; “low/high”; and “low/low” respectively. The first and second excipient sets can be dosed in an aqueous solution such as an aqueous ethanol solution.

In another embodiment the compound is supersaturated after dosing with the excipients. In a further embodiment supersaturation is obtained by increasing and decreasing the temperature of the receptacle. In some embodiments the receptacle is shaken after dosing. In another embodiment the receptacle is centrifuged after dosing. In yet another embodiment the receptacle is sonicated after dosing.

In some embodiments after the addition of the excipients the receptacles are analyzed to determine whether a precipitate has formed in the receptacle. Such analyses may be performed by light obscuration, visual observation or light microscopy. Other analytical techniques for analyzing solids, such as Raman spectroscopy or diffraction x-ray powder diffraction may also be utilized.

In another aspect of the invention, methods are provided which comprise performing a concentration gradient screen. In such a screen, varying amounts of compound are dosed into receptacles in a collection or series of arrays. This includes, but is not limited to, dosing from a minimum amount of solid to a maximum amount of solid. In another embodiment, one may dose increasing concentrations of the compound in an organic solvent along a series of receptacles. The compound can be dosed as a solid or in an organic solvent wherein the compound and organic solvent can be in the form of a solution. The screen then includes dosing into each set of receptacles a first and second excipient set selected from the ratios discussed above. After dosing with both the compound and the excipients, the receptacles arrays are analyzed for the presence of a precipitate. The methods further includes analyzing each receptacle for the presence of a precipitate; classifying the receptacles based on the presence of a precipitate to identify a solubility limit of the composition in the receptacle. Based on which receptacles in the gradient array first shows a precipitate, going from minimum to maximum concentration, a solubility limit of the combination can be estimated or identified. In another embodiment, the combination with the highest solubility is identified.

The active pharmaceutical ingredient and initial candidate first and second excipient sets can be dosed in an aqueous solution such as an aqueous ethanol solution.

The screening process may be repeated using the candidate compound excipient combination identified from the gradient screen, for example, the one with the highest solubility, as an input. One can then screen for formulations with additional excipients in the same manner as with a compound.

In another embodiment the compound is supersaturated after dosing with the excipients.

In other embodiments, supersaturation is obtained by increasing and decreasing the temperature of the receptacle. In some embodiments the receptacle is shaken after dosing. In another embodiment the receptacle is centrifuged after dosing. In yet another embodiment the receptacle is sonicated after dosing.

In yet a further embodiment the receptacles selected as desirable for further concentration gradient screening include, but are not limited to, those where the combination of compounds and excipients result in no precipitate, or where the precipitate which has formed is minimal compared with other receptacles.

In many embodiments the precipitate formation is evaluated using analytical methods including, but not limited to: light obscuration, visual observation, or optical microscopy.

The methods of the invention may be used to decrease the likelihood of failure due to dissolution limited absorption. These methods may also be used to select suitable candidate compound excipient combinations for development. The term “candidate compound excipient combination” or “combination” as used herein refers to a composition comprising a compound and at least two excipient sets. In some embodiments, the combination exhibits favorable solubility properties.

Using similar methodology the development of appropriate formulations for delivery of a low solubility compound through an aqueous delivery vehicle with multiple synergistic excipients is possible. Such a screen can be conducted on limited materials in as little as a few days, or a more detailed screen can be used to elucidate previously undiscovered synergies to maximize concentration in solution while minimizing excipient load.

Although presented in this example as a binary excipient formulation development, useful supersaturation effects without the use of large excipient loads may be possible using further multi-component systems that can be rapidly screened using this method.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

The invention will hereinafter be described by way of the following non-limiting Examples.

In the Examples below a liquid handling robot was used to develop a 96 wellplate method for testing different surfactant/excipient combinations with varied concentrations using carbamazepine (CBZ) as a model API. Binary mixes of 7 polymers, 11 surfactants, and an inclusion compound using four possible concentration combinations (High-High, High-Low, Low-High, and Low-Low) were dosed in an aqueous media at 55° C. with a fixed CBZ concentration. Wellplates were cooled to room temperature where CBZ was supersaturated with respect to the initial media. Crystallization was monitored by an obscuration method at 0, 24 h, and 7 d. Based on these results, a wellplate with a concentration gradient of CBZ using promising excipients from the initial study was designed and the results used to determine the upper bound of CBZ supersaturation in water at both 0 and after 1 d. Scale-up of formulations was conducted and thermodynamic solubility with respect to the CBZ hydrate was determined. The solubilities were compared to the results of the screen.

Aqueous formulations of CBZ in both 5% ethanol and water alone as the base media combined with a number of different polymers, an inclusion compound, and surfactants showed resistance to crystallization and improved solubility compared to the CBZ hydrate in the initial media. In water only, two formulation candidates (PVP K29/32 and HPβCD (5.0 w/v %/1.0 w/v %) and HPMC and cetrimide (0.75 w/v %/1.0 w/v %) had thermodynamic equilibrium solubilities that were 8 and 12 times that of the CBZ hydrate.

The semi-automated method was effective for developing aqueous formulations with improved API solution concentration and useful supersaturation. Although presented in this example as a binary formulation development, useful supersaturation effects without the use of large excipient loads may be possible using further multi component systems that can be rapidly screened using this method.

### EXAMPLES

### Example 1

Determining Candidate Compound Excipient Combinations

Based on High-High, High-Low, Low-High, and Low-Low excipient-surfactant and excipient-inclusion compound combinations, 336 experiments (including 6 repeats) were conducted in four 96-well×300 μL polystyrene flatbottom microplates. Stock solutions of the excipients, surfactants, and HPβCD were made at concentrations of 4 mg/mL.

Carbamazepine (CBZ)-ethanol solution (1 mg/mL; 80 μL; 80 μg) was dispensed into wells in the first three and last four rows of each microplate plus well D5 (control). The microplates were placed on a centrivap, and the ethanol was stripped in vacuuo at ambient temperature for ˜1 hour. Based on the experimental design, excipient, surfactant, and HPBCD solutions (5% Ethanol; 4 mg/mL), either 25 μL (0.10 mg) or 80 μL (0.32 mg) were then dispensed into the wells to provide w/w ratios of excipient, surfactant, and inclusion compound to API of 1.25 or 4.0 in the High-High, High-Low, Low-High, and Low-Low combinations. The wells were topped off with 5% Ethanol to give 200 μL total liquid charge. The 5% ethanol (200 μL) was also charged to wells D5 (CBZ control) and D7 (solvent only).

The microplates were capped with a virgin Teflon plate and the microplates were agitated on a shaker-incubator at ˜55° C. for ˜1 hour at ˜200 rpm. The microplate assemblies were cooled to room temperature on a bench for ˜1 hour before removing the Teflon plates and replacing with 2-mil TFE film. Sonication of the covered plates was conducted in a sonic bath for ˜5 min. followed by centrifugation for ˜2 minutes.

The microplates were inverted once with the film in place (to remove water condensate interference; before scanning through the film at intervals of time (time=0, 24 h and 7 d) on a UV-VIS microplate reader (600-900 nm, 2-nm step size; transmittance mode). Using a 5% ethanol blank, a pass-fail criterion was employed based on a cut-off of ˜86% transmittance between 750 and 800 nm, i.e., any transmittance below ˜86% was rejected.

The sonication-centrifugation steps were performed to limit perimeter crystallization interfering with analysis by the microplate reader. Optical microscopy was also used to confirm the presence of solids (Table 2).

TABLE 2
Example of Results from Initial Well Plate Screening

| Well | Visual: | MR | OM | Well | Visual: | MR | OM |
|---|---|---|---|---|---|---|---|
| B4 | NS | Pass | Pass | F4 | SM | Fail | — |
| B5 | SM | Pass | ~large tabs | F5 | SM | Fail | — |
| B6 | SM | Fail | — | F6 | SM | Fail | — |
| B7 | SM | Fail | — | F7 | SM | Fail | — |
| B8 | SM | Fail | — | F8 | SMP | Pass | P solids |
| B9 | SM | Fail | — | F9 | SM | Pass | P solids |
| B10 | SMP | Pass | P solids | F10 | SM | Fail | Tiny S's: |
| | | | | | | | center |
| B11 | SMP | Pass | P solids | F11 | SM | Fail | — |
| B12 | SMP | Pass | P solids | F12 | SM | Fail | — |

P = perimeter;
S = solids;
SM = solids with morphology;
NS = no solids;
tabs = tabulars;
MR = microplate reader;
OM = optical microscopy

Of 336 experiments conducted, 10 candidates were identified that yielded stable solutions after one week. The successful candidates for stabilizing CBZ at 400 μg/mL concentration (0.04 w/v %) were: PVPK29/32 (0.32 mg; 0.16 w/v %) and HPBCD (0.32; 0.16); PVPK29/32 (0.10; 0.05) and RH40 (0.10; 0.05); PolyOx (0.10; 0.05) and TPGS (0.32; 0.16); PVPVA64 (0.32; 0.16) and Cetrimide (0.10; 0.05); HPMC (0.32; 0.16) and Cetrimide (0.32; 0.16); PEG4000 (0.32; 0.16) and Tween 80 (0.10; 0.05); PVPVA64 (0.10; 0.05) and Tween 20 (0.32; 0.16); PVPVA64 (0.10; 0.05) and SDS (0.32; 0.16); HPC (0.10; 0.05) and RH40 (0.10; 0.05); PolyOx (0.10; 0.05) and SML (0.32; 0.16).

### Example 2

Gradient Concentration Study in 5% Ethanol

A kinetic gradient study was conducted in 5% ethanol with the 10 candidates identified in Example 1. CBZ-Ethanol solution was dispensed into 10 columns of wells in a 96-well microplate as follows: row A (80 μL @ 1 mg/mL; 80 μg API); row B (100 μL @ 2 mg/mL; 200 μg); row C (100 μL @ 4 mg/mL; 400 μg); row D (100 μL @ 6 mg/mL; 600 μg); row E (100 μL @ 8 mg/mL; 800 μg); row F (100 μL @ 20 mg/mL; 2 mg); row G (2×100 μL @ 20 mg/mL; 4 mg); row H (4×100 μL @ 20 mg/mL; 8 mg). Thus, a first-pass filling of the wellplate was conducted. The wellplate was placed on a centrivap and the solvent stripped at ambient temperature for ˜4 hours. The wells in rows G and H were refilled with CBZ solution, and the solvent stripped at ambient temperature for ˜1 hour, resulting in a net CBZ charge to rows G and H of 4 mg per well. This procedure was repeated twice for row H, resulting in a net CBZ charge of 8 mg per well. Thus, the final w/v % CBZ by row (200 μL liquid) was 0.04, 0.1, 0.2, 0.3, 0.4, 1.0, 2.0, and 4.0. To the wells in columns 1 through 10 were charged the appropriate excipient, surfactant, and HPBCD solutions (4 mg/mL; 25 or 80 μL 5% Ethanol solution) that were previously determined in Example 1. Additional 5% Ethanol solvent was added to the wells to give 200 μL total liquid. The solvent (200 μL) was also charged to wells in column 11 (API control) and 12 (solvent only).

The microplate was capped with a virgin Teflon plate and agitated on a shaker-incubator at ˜55° C. for ˜1 hour at ˜200 rpm. The plate assembly was cooled to room temperature on a bench for ˜1 hour before removing the Teflon plate and replacing with 2-mil TFE film. Sonication of the covered plates was conducted in a sonic bath for ˜5 min. followed by centrifugation for ˜2 minutes. The sonication was performed to encourage crystallite formation, and centrifugation was employed to force as much of the solid to the center of each well in preparation for UV-VIS scanning.

The microplate was inverted before scanning through the film on a UV-VIS microplate reader (600-900 nm, 2-nm step size; transmittance). The plate was then removed, and optical microscopy was used to scan through the TFE film at day 0. Solids with morphology were observed in every well in the first three rows of columns 1 through 11. Gross amounts of solid were observed in the remaining rows. The solvent blank column showed no solid, as expected. Results are shown in Table 3.

TABLE 3
Example Results from a 5% Ethanol Gradient Concentration study

| Well | PLM | Well | PLM |
|---|---|---|---|
| A1 | SM; a few tiny aciculars | E1 | gross S |
| A2 | SM; a few tiny aciculars | E2 | gross S |
| A3 | SMP; small fibrous | E3 | gross S |
| A4 | SMP; aciculars | E4 | gross S |
| A5 | SM; tiny aciculars | E5 | gross S |
| A6 | SM; tiny aciculars | E6 | gross S |
| A7 | SMP; appreciable fibrous | E7 | gross S |
| A8 | SM; a few tiny blades | E8 | gross S |
| A9 | SMP; a few tiny fibrous | E9 | gross S |
| A10 | SMP; appreciable aciculars | E10 | gross S |
| A11 | SM; a few tiny B/E particles | E11 | gross S |
| A12 | NS | E12 | NS |

S = solids;
NS = no solids;
SM = solids with morphology;
P = perimeter;
B = birefringent;
E = extinguishable

### Example 3

Gradient Concentration Study in Water

Using similar techniques to those described in Example 2, a kinetic gradient study was conducted in water with the same 10 Excipient candidates identified in Example 1. The low and high concentrations of the excipients were set at 1.0 (low) and 5.0 w/v % (high). The exception to this was HPMC, which was set at 0.75% (high) due to its ability to produce relatively high viscosity solutions in water. The surfactants and HPBCD were set at 0.1 (low) and 1.0 w/v % (high). The CBZ gradient was set as follows: row 1 (0.01 w/v %); row 2 (0.02); row 3 (0.04%); row 4 (0.08%); row 5 (0.16%); row 6 (0.32%); row 7 (0.64%). A sonicated and an unsonicated wellplate were employed in this example. A UV-VIS scan was performed, and optical microscopy was used to scan through the TFE film at days 0 and 1 (Table 4 and 5).

TABLE 4
Example Results of Ambient Gradient Concentration Study in
Water (Day 0)

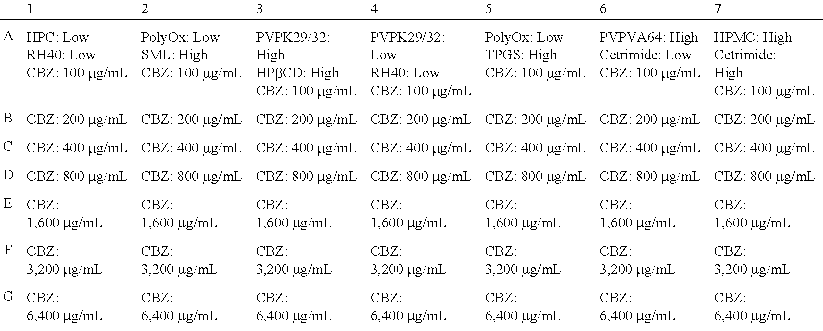

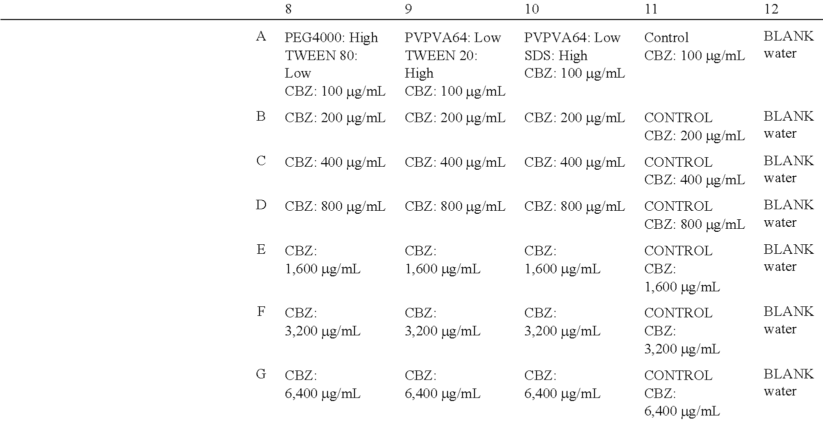

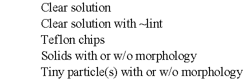

TABLE 5
Example Results of Ambient Gradient Concentration Study in
Water (Day 1)

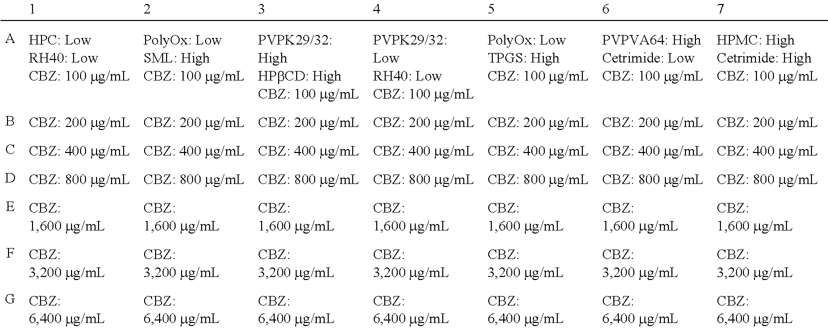

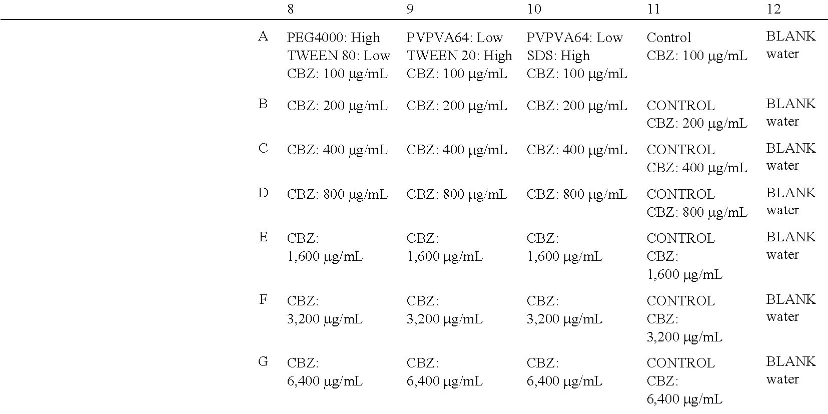

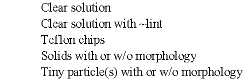

In general, the sonicated microplate had greater kinetic solubility than the unsonicated microplate. Thus, for low/low HPC and RH40, the kinetically stable API concentrations were 0.02 (sonicated) vs. 0.01 w/v % (unsonicated); low/high PolyOx and SML were 0.04 vs. 0.04%; high/high PVPK29/32 and HPβCD were 0.08 vs. 0.02%; low/low PVPK29/32 and RH40 were 0.02 vs. 0.02%; low/high; low/high PolyOx and TPGS were 0.04 vs. 0.02%; high/low PVPVA64 and cetrimide were 0.04 vs. 0.04%; high/high HPMC and cetrimide were 0.08 vs. 0.04%; high/low PEG4000 and Tween 80 were 0.02 vs. 0.02%; low/high PVPVA64 and Tween 20 were 0.04 vs. 0.02%; PVPVA64 and SDS were 0.08 vs. 0.04%; the control was 0.02% vs. 0.02%.

The equilibrium solubility and the resulting solid form were determined to assess supersaturation stability. Equilibrium solubility was determined based on the excipient-surfactant and excipient-HPβCD ratios above, a 20:1 scale-up was performed. Teflon “X” stir bars were added to 2 dram vials, and CBZ solid plus the appropriate volumes of excipient, surfactant, and HPβCD aqueous solutions (total: 4.0 mL each) were charged. Stir speed was set at ˜700 rpm, and the resulting slurries were stirred for at least 1 day at ambient temperature out of light. CBZ dihydrate was added, the slurries were stirred for ˜1 day at ambient temperature, centrifuged, and the supernatants filtered on 0.45-μm GHP. The associated solids were blotted on Whatman paper and analyzed by Bruker transmission X-ray powder diffraction (XRPD). The final results are shown in Table 5.

TABLE 5
Results of Gradient Screening

| | | | | | | T = 0 | T = 1 d |
|---|---|---|---|---|---|---|---|
| | | | | | Eq. Sol. | Range | Range |
| Polymer | w/v % | Surfactant | w/v % | XRPD Result | (μg/mL) | (μg/mL) | (μg/mL) |
| HPC | 1.0 | RH40 | 0.1 | dihydrate | 184 | 200-400 | 100-200 |
| PolyOx | 1.0 | SML | 1.0 | dihydrate | 553 | Mixed | 400-800 |
| | | | | | | results | |
| PVPK29/32 | 5.0 | HPβCD | 1.0 | dihydrate | 1027 | 800-1600 | 200-400 |
| PVPK29/32 | 1.0 | RH40 | 0.1 | dihydrate | 212 | 200-400 | 200-400 |
| PolyOx | 1.0 | TPGS | 1.0 | III + dihydrate | 617 | 400-800 | 200-400 |
| PVPVA64 | 5.0 | Cetrimide | 0.1 | dihydrate | 525 | 800-1600 | 400-800 |
| HPMC | 0.75 | Cetrimide | 1.0 | III + dihydrate | 1535 | 400-800 | 400-800 |
| PEG4000 | 5.0 | TWEEN 80 | 0.1 | III + dihydrate | 307 | 400-800 | 200-400 |
| PVPVA64 | 1.0 | TWEEN 20 | 1.0 | dihydrate | 339 | 400-800 | 200-400 |
| PVPVA64 | 1.0 | SDS | 1.0 | dihydrate | 842 | 400-800 | 400-800 |

It will be appreciated by persons skilled in the art that numerous variations and/or modifications may be made to the invention as shown in the specified embodiments without departing from the spirit or scope of the invention as broadly described. The present embodiments are, therefore, to be considered in all respects as illustrative and not restrictive.

Any discussion of documents, acts, materials, devices, articles or the like which was included in the present specification is solely for the purpose of providing a context for the present invention. It is not to be taken as an admission that any or all of these matters form part of the prior art base or were common general knowledge in the field relevant to the present invention as it existed in any country before the priority date of each claim of this application.

1. A method of screening for candidate compound-excipient combinations comprising:
dosing a compound into each receptacle of a collection of receptacles;
dosing a first set of excipients and a second set of excipients into said receptacles wherein said dosing creates multiple combinations of solutions of the first excipient set and the second excipient set within said receptacles;
analyzing each receptacle for the presence of a precipitate; and
classifying the receptacles based on the presence of a precipitate.

2. The method of claim 1 wherein said compound is selected from the group consisting of an active pharmaceutical ingredient, a nutraceutical, or an agricultural active substance.

3. The method of claim 1 wherein said receptacle is a well plate, a vial, or a test tube.

4. The method of claim 3 wherein said receptacle is a well plate.

5. The method of claim 1 wherein said compound is dosed as a solid.

6. The method of claim 1 wherein said compound is dosed in an organic solvent.

7. The method of claim 6 wherein said compound and organic solvent form a solution.

8. The method of claim 1 wherein said receptacles are classified as either having precipitate or not having precipitate.

9. The method of claim 1 wherein said receptacles are classified based on the relative amount of precipitate present.

10. The method of claim 6 further comprising the step of removing the organic solvent prior to dosing with the first and second excipient sets.

11. The method of claim 1 wherein at least one set of excipients includes a set of polymers.

12. The method of claim 1 wherein at least one set of excipients includes a set of surfactants.

13. The method of claim 1 wherein at least one set of excipients includes a set of inclusion compounds.

14. The method of claim 1 wherein the concentration of said first and second excipient sets are in ratios relative to their amounts dosed in the ratios of high/high, high/low, low/high and low/low combinations of said first excipient/second excipient sets wherein the high concentration and the low concentrations are fixed for a set of receptacles.

15. The method of claim 1 wherein precipitation is analyzed in said receptacles using light obscuration or visual observation.

16. The method of claim 1 wherein precipitation is analyzed in said receptacles using optical microscopy.

17. The method of claim 10 wherein said solvent is removed by evaporation.

18. The method of claim 1 wherein said first and second excipient sets are dosed in an aqueous solution.

19. The method of claim 18 wherein said first and second excipient sets are dosed in an aqueous ethanol solution.

20. The method of claim 1 wherein said compound is supersaturated after dosing with the excipients.

21. The method of claim 20 wherein supersaturation is obtained by increasing and then decreasing the temperature of said receptacle.

22. The method of claim 1 wherein said receptacle is shaken after dosing.

23. The method of claim 1 wherein said receptacle is centrifuged after dosing.

24. The method of claim 1 wherein said receptacle is sonicated after dosing.

25. The method of claim 1 further comprising performing a concentration gradient analysis comprising:
dosing said compound into a second set of receptacles wherein said compound is dosed from a minimum to a maximum amount of compound;
dosing into each set of receptacles a first and second excipient set selected from the ratios identified in claim 14;
analyzing each receptacle for the presence of a precipitate;
classifying the receptacles based on the presence of a precipitate to identify a solubility limit of the composition in the receptacle.
